(19) United States
(12) Patent Application Publication
(10) Pub. No.: US 2019/0315920 A1
(43) Pub. Date: Oct. 17, 2019

(54) SHARP POLYMER AND CAPACITOR

(71) Applicant: Capacitor Sciences Incorporated, Menlo Park, CA (US)

(72) Inventors: Barry K. SHARP, Redwood City, CA (US); Paul FURUTA, Sunnyvale, CA (US); Pavel Ivan LAZAREV, Menlo Park, CA (US)

(21) Appl. No.: 16/457,169

(22) Filed: Jun. 28, 2019

Related U.S. Application Data
(62) Division of application No. 15/043,247, filed on Feb. 12, 2016.

(51) Int. Cl. C08G 73/06; H01G 4/005; H01G 4/32; H01G 4/14; C07D 471/22

(52) U.S. Cl. CPC C08G 73/0688; H01G 4/005; C07D 471/22; H01G 4/14; H01G 4/32

(57) ABSTRACT

A meta-dielectric film usable in a capacitor includes composite molecules with a resistive envelope built with alkyl oligomeric single chain or branched chain oligomers having carbo-hydrogen or carbo-fluoro composition and a polarizable core molecular fragment inside the resistive envelope. The polarizable core has an electronic or ionic type of polarizability provided by electronic conductivity of the core molecular fragment or limited mobility of ionic parts of the core molecular fragment.

### FIELD OF THE INVENTION

The present invention relates generally to passive components of electrical circuit and more particularly to a composite organic compound and capacitor based on this material and intended for energy storage.

### BACKGROUND OF THE INVENTION

A capacitor is a passive electronic component that is used to store energy in the form of an electrostatic field, and comprises a pair of electrodes separated by a dielectric layer. When a potential difference exists between the two electrodes, an electric field is present in the dielectric layer. An ideal capacitor is characterized by a single constant value of capacitance, which is a ratio of the electric charge on each electrode to the potential difference between them. For high voltage applications, much larger capacitors have to be used.

One important characteristic of a dielectric material is its breakdown field. This corresponds to the value of electric field strength at which the material suffers a catastrophic failure and conducts electricity between the electrodes. For most capacitor geometries, the electric field in the dielectric can be approximated by the voltage between the two electrodes divided by the spacing between the electrodes, which is usually the thickness of the dielectric layer. Since the thickness is usually constant it is more common to refer to a breakdown voltage, rather than a breakdown field. There are a number of factors that can dramatically reduce the breakdown voltage. In particular, the geometry of the conductive electrodes is important factor affecting breakdown voltage for capacitor applications. In particular, sharp edges or points hugely increase the electric field strength locally and can lead to a local breakdown. Once a local breakdown starts at any point, the breakdown will quickly “trace” through the dielectric layer until it reaches the opposite electrode and causes a short circuit.

Breakdown of the dielectric layer usually occurs as follows. Intensity of an electric field becomes high enough to “pull” electrons from atoms of the dielectric material and makes them conduct an electric current from one electrode to another. Presence of impurities in the dielectric or imperfections of the crystal structure can result in an avalanche breakdown as observed in semiconductor devices.

Another of important characteristic of a dielectric material is its dielectric permittivity. Different types of dielectric materials are used for capacitors and include ceramics, polymer film, paper, and electrolytic capacitors of different kinds. The most widely used polymer film materials are polypropylene and polyester. Increasing dielectric permittivity allows for increasing volumetric energy density, which makes it an important technical task.

An ultra-high dielectric constant composite of polyaniline, PANI-DBSA/PAA, was synthesized using in situ polymerization of aniline in an aqueous dispersion of poly-acrylic acid (PAA) in the presence of dodecylbenzene sulfonate (DBSA) (see, Chao-Hsien Hoa et al., “High dielectric constant polyaniline/poly(acrylic acid) composites prepared by in situ polymerization”, Synthetic Metals 158 (2008), pp. 630-637). The water-soluble PAA served as a polymeric stabilizer, protecting the PANI particles from macroscopic aggregation. A very high dielectric constant of about 2.0×10⁵ (at 1 kHz) was obtained for the composite containing 30% PANI by weight. Influence of the PANI content on the morphological, dielectric and electrical properties of the composites was investigated. Frequency dependence of dielectric permittivity, dielectric loss, loss tangent and electric modulus were analyzed in the frequency range from 0.5 kHz to 10 MHz. SEM micrograph revealed that composites with high PANI content (i.e., 20 wt. %) consisted of numerous nano-scale PANI particles that were evenly distributed within the PAA matrix. High dielectric constants were attributed to the sum of the small capacitors of the PANI particles. The drawback of this material is a possible occurrence of percolation and formation of at least one continuous electrically conductive channel under electric field with probability of such an event increasing with an increase of the electric field. When at least one continuous electrically conductive channel (track) through the neighboring conducting PANI particles is formed between electrodes of the capacitor, it decreases a breakdown voltage of such capacitor.

Colloidal polyaniline particles stabilized with a water-soluble polymer, poly(N-vinylpyrrolidone) [poly(1-vinylpyrrolidin-2-one)], have been prepared by dispersion polymerization. The average particle size, 241+50 nm, have been determined by dynamic light scattering (see, Jaroslav Stejskal and Irina Sapurina, “Polyaniline: Thin Films and Colloidal Dispersions (IUPAC Technical Report)”, Pure and Applied Chemistry, Vol. 77, No. 5, pp. 815-826 (2005).

Single crystals of doped aniline oligomers are produced via a simple solution-based self-assembly method (see, Yue Wang, et. al., “Morphological and Dimensional Control via Hierarchical Assembly of Doped Oligoaniline Single Crystals”, J. Am. Chem. Soc. 2012, 134, pp. 9251-9262). Detailed mechanistic studies reveal that crystals of different morphologies and dimensions can be produced by a “bottom-up” hierarchical assembly where structures such as one-dimensional (1-D) nanofibers can be aggregated into higher order architectures. A large variety of crystalline nanostructures, including 1-D nanofibers and nanowires, 2-D nanoribbons and nanosheets, 3-D nanoplates, stacked sheets, nanoflowers, porous networks, hollow spheres, and twisted coils, can be obtained by controlling the nucleation of the crystals and the non-covalent interactions between the doped oligomers. These nanoscale crystals exhibit enhanced conductivity compared to their bulk counterparts as well as interesting structure-property relationships such as shape-dependent crystallinity. Furthermore, the morphology and dimension of these structures can be largely rationalized and predicted by monitoring molecule-solvent interactions via absorption studies. Using doped tetra-aniline as a model system, the results and strategies presented in this article provide insight into the general scheme of shape and size control for organic materials.

Thus, materials with high dielectric permittivity which are based on composite materials and containing polarized particles (such as PANI particles) may demonstrate a percolation phenomenon. The formed polycrystalline structure of layers has multiple tangling chemical bonds on borders between crystallites. When the used material with high dielectric permittivity possesses polycrystalline structure, a percolation may occur along the borders of crystal grains.

Hyper-electronic polarization of organic compounds is described in greater detail in Roger D. Hartman and Herbert A. Pohl, “Hyper-electronic Polarization in Macromolecular Solids”, Journal of Polymer Science: Part A-1 Vol. 6, pp. 1135-1152 (1968). Hyper-electronic polarization may be viewed as the electrical polarization external fields due to the pliant interaction with the charge pairs of excitons, in which the charges are molecularly separated and range over molecularly limited domains. In this article four polyacene quinone radical polymers were investigated. These polymers at 100 Hz had dielectric constants of 1800-2400, decreasing to about 58-100 at 100,000 Hz. Essential drawback of the described method of production of material is use of a high pressure (up to 20 kbars) for forming the samples intended for measurement of dielectric constants.

Capacitors as energy storage device have well-known advantages versus electrochemical energy storage, e.g. a battery. Compared to batteries, capacitors are able to store energy with very high power density, i.e. charge/recharge rates, have long shelf life with little degradation, and can be charged and discharged (cycled) hundreds of thousands or millions of times. However, capacitors often do not store energy in small volume or weight as in case of a battery, or at low energy storage cost, which makes capacitors impractical for some applications, for example electric vehicles. Accordingly, it may be an advance in energy storage technology to provide capacitors of higher volumetric and mass energy storage density and lower cost.

### SUMMARY

The present disclosure provides a dielectric film comprising composite molecules with a resistive envelope built with oligomers having a composition of hydrocarbon (saturated and/or unsaturated), fluorocarbon, siloxane, and/or polyethyleneglycol as linear or branched chains and a polarizable core molecular fragment inside the resistive envelope, wherein the polarizable core has an electronic or ionic type of polarizability provided by electronic conductivity of the core molecular fragment or limited mobility of ionic parts of the core molecular fragment.

In one aspect, the aforementioned composite organic compound may be used in a capacitor as a dielectric film between two electrodes. This type of composite organic compound is referred to herein as a “Sharp polymer”. A dielectric film made with a Sharp polymer is one type of material referred to herein as a “meta-dielectric”. A capacitor made using a meta-dielectric between two electrodes is referred to herein as a “meta-capacitor”.

In one implementation, a meta-dielectric film is made of a Sharp polymer in the form of a composite organic compound characterized by polarizability and resistivity and having the following general structural formula:

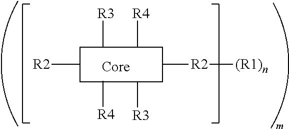

Where Core is an aromatic polycyclic conjugated molecule. This molecule has flat anisometric form and self-assembles by pi-pi stacking in a column-like supramolecule. The substitute R1 provides solubility of the organic compound in a solvent. The parameter n is number of substitutes R1, which is equal to 0, 1, 2, 3, 4, 5, 6, 7 or 8. The substitute R2 is an electrically resistive substitute located in terminal positions, which provides resistivity to electric current and comprises hydrocarbon (saturated and/or unsaturated), fluorocarbon, siloxane, and/or polyethyleneglycol as linear or branched chains. The substitutes R3 and R4 are substitutes located on side (lateral) positions (terminal and/or bay positions) comprising one or more ionic groups from a class of ionic compounds that are used in ionic liquids connected to the aromatic polycyclic conjugated molecule (Core), either directly, e.g., with direct bound SP2-SP3 carbons, or via a connecting group. The parameter m is a number of the aromatic polycyclic conjugated molecules in the column-like supramolecule, which is in a range from 3 to 100,000.

In another aspect, a meta-dielectric film capacitor includes two metal electrodes and a meta-dielectric film between the two electrodes. The meta-dielectric film comprises composite molecules with a resistive envelope built with oligomers having a composition of hydrocarbon (saturated and/or unsaturated), fluorocarbon, siloxane, and/or polyethylene glycol as linear or branched chains and a polarizable core molecular fragment inside the resistive envelope, wherein the polarizable core has an electronic or ionic type of polarizability provided by electronic conductivity of the core molecular fragment or limited mobility of ionic parts of the core molecular fragment. The two electrodes may be positioned parallel to each other and may be rolled or flat and planar.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

FIG. 1A is a cross-sectional schematic diagram depicting a meta-capacitor in accordance with aspects of the present disclosure.

FIG. 1B is a three-dimensional schematic view of a coiled meta-capacitor in accordance with aspects of the present disclosure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure provides a Sharp polymer in the form of a composite organic compound. In one embodiment of the composite organic compound, the aromatic polycyclic conjugated molecule (Core) comprises rylene fragments. In another embodiment of the composite organic compound, the rylene fragments are selected from structures 1 to 21 as given in Table 1.

TABLE 1
Examples of the polycyclic organic molecule (Core) comprising rylene fragments

| | |
|---|---|
| 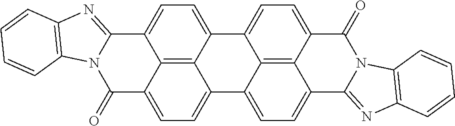 [SMILES: O=c1c2ccc3c4ccc5c6c(ccc(c7ccc(c2c37)c2nc3ccccc3n12)c46)c(=O)n1c2ccccc2nc51] | 1 |
| 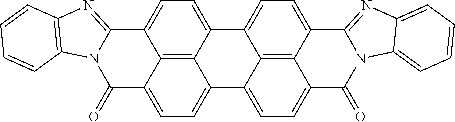 [SMILES: O=c1c2ccc3c4ccc5c(=O)n6c7ccccc7nc6c6ccc(c7ccc(c2c37)c2nc3ccccc3n12)c4c56] | 2 |
| 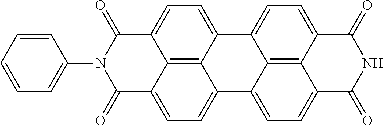 [SMILES: O=C1NC(=O)c2ccc3c4ccc5c6c(ccc(c7ccc1c2c73)c64)C(=O)N(c1ccccc1)C5=O] | 3 |
| 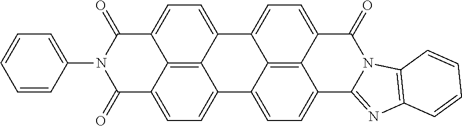 [SMILES: O=C1c2ccc3c4ccc5c(=O)n6c7ccccc7nc6c6ccc(c7ccc(c2c37)C(=O)N1c1ccccc1)c4c56] | 4 |
| 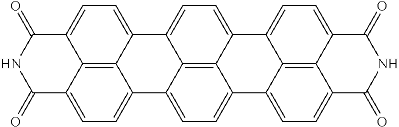 [SMILES: O=C1NC(=O)c2ccc3c4ccc5c6ccc7c8c(ccc(c9ccc(c%10ccc1c2c%103)c4c95)c86)C(=O)NC7=O] | 5 |
| 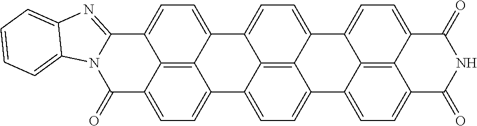 [SMILES: O=C1NC(=O)c2ccc3c4ccc5c6ccc7c8c(ccc(c9ccc(c%10ccc1c2c%103)c4c95)c68)c(=O)n1c2ccccc2nc71] | 6 |
| 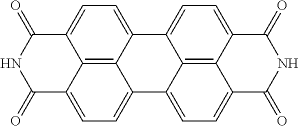 [SMILES: O=C1NC(=O)c2ccc3c4ccc5c6c(ccc(c7ccc1c2c73)c64)C(=O)NC5=O] | 7 |
| 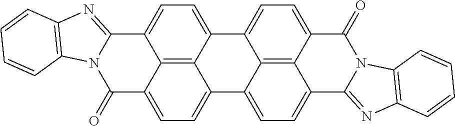 [SMILES: O=c1c2ccc3c4ccc5c6c(ccc(c7ccc(c2c37)c2nc3ccccc3n12)c46)c(=O)n1c2ccccc2nc51] | 8 |
| 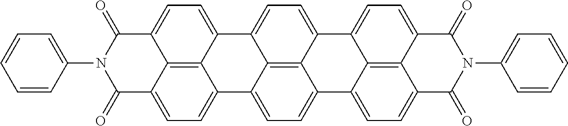 [SMILES: O=C1c2ccc3c4ccc5c6ccc7c8c(ccc(c9ccc(c%10ccc(c2c3%10)C(=O)N1c1ccccc1)c4c59)c86)C(=O)N(c1ccccc1)C7=O] | 9 |
| 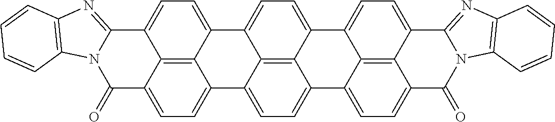 [SMILES: O=c1c2ccc3c4ccc5c6ccc7c(=O)n8c9ccccc9nc8c8ccc(c9ccc(c%10ccc(c2c3%10)c2nc3ccccc3n12)c4c59)c6c78] | 10 |
| 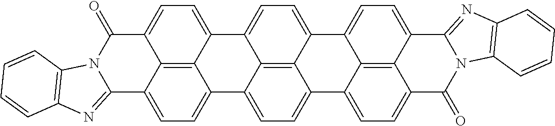 [SMILES: O=c1c2ccc3c4ccc5c6ccc7c8c(ccc(c9ccc(c%10ccc(c2c3%10)c2nc3ccccc3n12)c4c95)c68)c(=O)n1c2ccccc2nc71] | 11 |
| 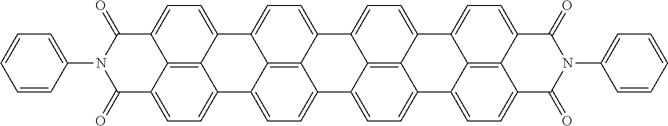 | 12 |
| 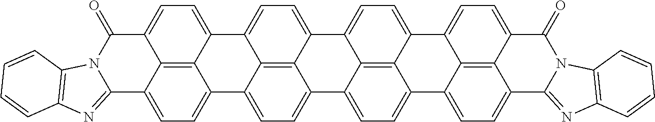 | 13 |
| 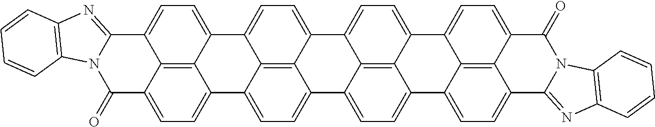 | 14 |
| 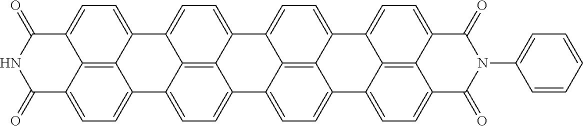 | 15 |
| 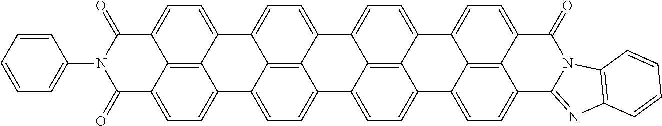 | 16 |
| 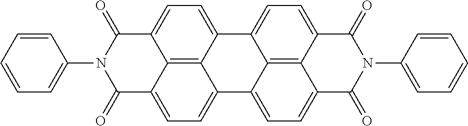 [SMILES: O=C1c2ccc3c4ccc5c6c(ccc(c7ccc(c2c37)C(=O)N1c1ccccc1)c64)C(=O)N(c1ccccc1)C5=O] | 17 |
| 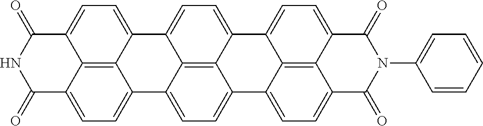 [SMILES: O=C1NC(=O)c2ccc3c4ccc5c6ccc7c8c(ccc(c9ccc(c%10ccc1c2c%103)c4c95)c86)C(=O)N(c1ccccc1)C7=O] | 18 |
| 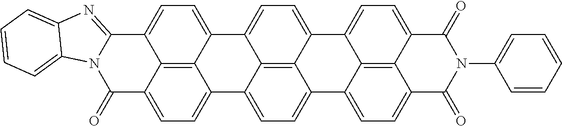 [SMILES: O=C1c2ccc3c4ccc5c6ccc7c(=O)n8c9ccccc9nc8c8ccc(c9ccc(c%10ccc(c2c3%10)C(=O)N1c1ccccc1)c4c59)c6c78] | 19 |
| 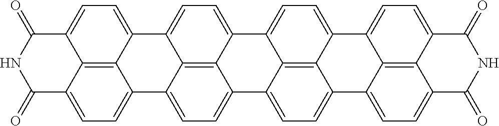 | 20 |
| 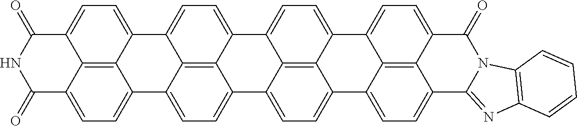 | 21 |

In another embodiment of the composite organic compound, the aromatic polycyclic conjugated molecule comprises an electro-conductive oligomer, such as a phenylene, thiophene, or polyacene quinine radical oligomer or combinations of two or more of these. In yet another embodiment of the composite organic compound, the electro-conductive oligomer is selected from structures 22 to 30 as given in Table 2, wherein I=2, 3, 4, 5, 6, 7, 8, 9, 10, 11 or 12, Z is ═O, ═S or ═NR5, and R5 is selected from the group consisting of unsubstituted or substituted C₁-C₁₈alkyl, unsubstituted or substituted C₂-C₁₈alkenyl, unsubstituted or substituted C₂-C₁₈alkynyl, and unsubstituted or substituted C₄-C₁₈aryl:

TABLE 2
Examples of the polycyclic organic molecule (Core) comprising
electro-conductive oligomer

| | |
|---|---|
| 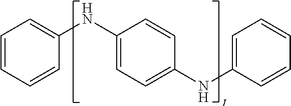 | 22 |
| 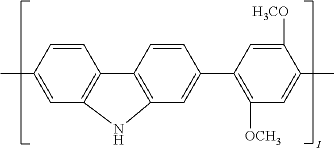 | 23 |
| 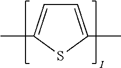 | 24 |
| 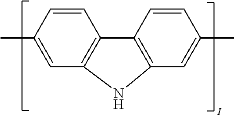 | 25 |
| 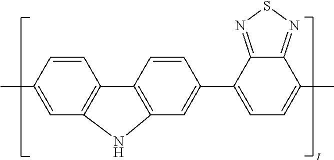 | 26 |
| 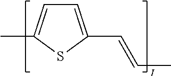 | 27 |
| 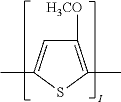 | 28 |
| 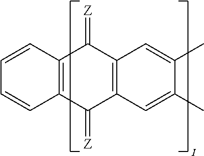 | 29 |
| 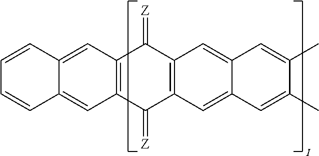 | 30 |

In some embodiments, the substitute providing solubility (R1) of the composite organic compound is C_{X}Q_{2X+1}, where X≥1 and Q is hydrogen (H), fluorine (F), or chlorine (Cl). In still another embodiment of the composite organic compound, the substitute providing solubility (R1) of the composite organic compound is independently selected from alkyl, aryl, substituted alkyl, substituted aryl, fluorinated alkyl, chlorinated alkyl, branched and complex alkyl, branched and complex fluorinated alkyl, branched and complex chlorinated alkyl groups, and any combination thereof, and wherein the alkyl group is selected from methyl, ethyl, propyl, butyl, iso-butyl and tert-butyl groups, and the aryl group is selected from phenyl, benzyl and naphthyl groups or siloxane, and/or polyethyleneglycol as linear or branched chains.

In one embodiment of the composite organic compound, the solvent is selected from benzene, toluene, xylenes, acetone, acetic acid, methylethylketone, hydrocarbons, chloroform, carbontetrachloride, methylenechloride, dichlorethane, chlorobenzene, alcohols, nitromethan, acetonitrile, dimethylforamide, 1,4-dioxane, tetrahydrofuran (THF), methylcyclohexane (MCH), and any combination thereof.

In some embodiments, at least one electrically resistive substitute (R2) of the composite organic compound is C_{X}Q_{2X+1}, where X≥1 and Q is hydrogen (H), fluorine (F), or chlorine (Cl). In another embodiment of the composite organic compound, at least one electrically resistive substitute (R2) is selected from the list comprising —(CH₂)ₙ—CH₃, —CH((CH₂)ₙCH₃)₂) (where n≥1), alkyl, aryl, substituted alkyl, substituted aryl, branched alkyl, branched aryl, and any combination thereof and wherein the alkyl group is selected from methyl, ethyl, propyl, butyl, I-butyl and t-butyl groups, and the aryl group is selected from phenyl, benzyl and naphthyl groups. In yet another embodiment of the composite organic compound.

In some embodiments, at least one electrically resistive substitute (R2) is selected from the group of alkyl, aryl, substituted alkyl, substituted aryl, fluorinated alkyl, chlorinated alkyl, branched and complex alkyl, branched and complex fluorinated alkyl, branched and complex chlorinated alkyl groups, and any combination thereof, and wherein the alkyl group is selected from methyl, ethyl, propyl, n-butyl, iso-butyl and tert-butyl groups, and the aryl group is selected from phenyl, benzyl and naphthyl groups or siloxane, and/or polyethyleneglycol as linear or branched chains.

In some embodiments, the substitute R1 and/or R2 is connected to the aromatic polycyclic conjugated molecule (Core) via at least one connecting group. The at least one connecting group may be selected from the list comprising the following structures: 31-41 as given in Table 3, where W is hydrogen (H) or an alkyl group.

TABLE 3
Examples of the connecting group

| | |
|---|---|
| —O— | 31 |
| 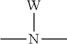 | 32 |
| 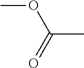 | 33 |
| 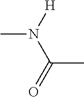 | 34 |
| 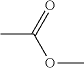 | 35 |
| 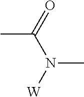 | 36 |
| 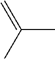 | 37 |
| 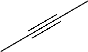 | 38 |
| 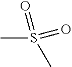 | 39 |
| 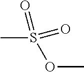 | 40 |
| 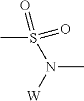 | 41 |

In some embodiments, the substitute R3 and/or R4 may be connected to the aromatic polycyclic conjugated molecule (Core) via at least one connecting group. The at least one connecting group may be selected from the list comprising CH₂, CF₂, SiR₂O, CH₂CH₂O, wherein R is selected from the list comprising H, alkyl, and fluorine. In another embodiment of the composite organic compound, the one or more ionic groups include at least one ionic group selected from the list comprising [NR₄], [PR₄] as cation and [—CO₂]⁻, [—SO₃]⁻, [—SR₅]⁻, [—PO₃R]⁻, [—PR₅]⁻ as anion, wherein R is selected from the list comprising H, alkyl, and fluorine.

The Sharp polymers have hyperelectronic or ionic type polarizability. “Hyperelectronic polarization may be considered due to the pliant interaction of charge pairs of excitons, localized temporarily on long, highly polarizable molecules, with an external electric field [.] (Roger D. Hartman and Herbert A. Pohl, “Hyper-electronic Polarization in Macromolecular Solids”, Journal of Polymer Science: Part A-1 Vol. 6, pp. 1135-1152 (1968)).” Ionic type polarization can be achieved by limited mobility of ionic parts of the tethered/partially immobilized ionic liquid or zwitterion (Q). Additionally, other mechanisms of polarization such as dipole polarization and monomers and polymers possessing metal conductivity may be used independently or in combination with hyper-electronic and ionic polarization in aspects of the present disclosure.

In another aspect, the present disclosure provides a meta-dielectric, wherein a meta-dielectric is a dielectric that includes one or more Sharp polymers in the form of a composite organic compound characterized by polarizability and resistivity having the following general structural formula, which is described in detail hereinabove:

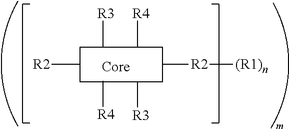

Further, characteristics of meta-dielectrics include a relative permittivity greater than or equal to 1,000 and resistivity greater than or equal to 10¹³ ohm/cm. Individually, the Sharp Polymers in a meta-dielectric may form column like supramolecular structures by pi-pi interaction. Said supramolecules of Sharp polymers allow formation of crystal structures of the meta-dielectric material. By way of using Sharp polymers in a dielectric material, polarization units are incorporated to provide the molecular material with high dielectric permeability. There are several mechanisms of polarization such as dipole polarization, ionic polarization, and hyper-electronic polarization of molecules, monomers and polymers possessing metal conductivity. All polarization units with the listed types of polarization may be used in aspects of the present disclosure. Further, Sharp polymers are composite materials which incorporate an envelope of insulating substituent groups that electrically isolate the supramolecules from each other in the dielectric crystal layer and provide high breakdown voltage of the energy storage molecular material. Said insulating substituent groups are resistive alkyl or fluro-alkyl chains covalently bonded to a polarizable core, forming the resistive envelope.

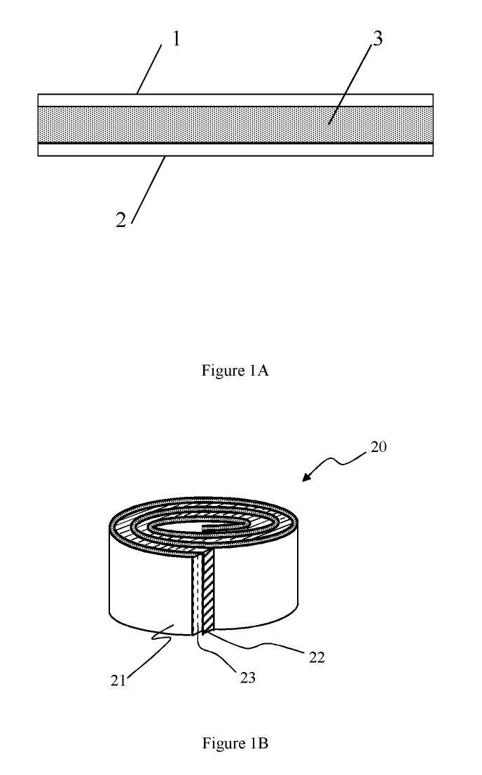

In another aspect, the present disclosure provides a meta-capacitor shown in FIG. 1A. The capacitor comprises a first electrode 1, a second electrode 2, and a meta-dielectric Film layer 3 disposed between said first and second electrodes. The electrodes may be flat and planar and positioned parallel to each other. In another embodiment the meta-dielectric Film capacitor, the electrodes 1, 2 are in the form of two rolled metal electrodes positioned parallel to each other with the meta-dielectric Film layer 3 sandwiched between them.

The electrodes 1, 2 may be flat and planar and positioned parallel to each other. Alternatively, the electrodes may be planar and parallel, but not necessarily flat, e.g., they may coiled, rolled, bent, folded, or otherwise shaped to reduce the overall form factor of the capacitor. It is also possible for the electrodes to be non-flat, non-planar, or non-parallel or some combination of two or more of these. By way of example and not by way of limitation, a spacing d between the electrodes 1, 2 which may correspond to the thickness of the meta-dielectric Film layer 3 may range from about 100 nm to about 10,000 m. As noted in Equation (2) above, the maximum voltage V_{bd} between the electrodes 1, 2 is approximately the product of the breakdown field and the electrode spacing d. For example, if, E_{bd}=0.1 V/nm and the spacing d between the electrodes 1, 2 is 10,000 microns (100,000 nm), the maximum voltage V_{bd} would be 100,000 volts.

The electrodes 1, 2 may have the same shape as each other, the same dimensions, and the same area A. By way of example, and not by way of limitation, the area A of each electrode 1, 2 may range from about 0.01 m² to about 1000 m². By way of example and not by way of limitation, for rolled capacitors, electrodes up to, e.g., 1000 m long and 1 m wide.

These ranges are non-limiting. Other ranges of the electrode spacing d and area A are within the scope of the aspects of the present disclosure.

If the spacing d is small compared to the characteristic linear dimensions of electrodes (e.g., length and/or width), the capacitance C of the capacitor may be approximated by the formula:

C=κε₀A/d, (3)

where ε₀ is the permittivity of free space (8.85×10⁻¹² Coulombs²/(Newton·meter²)) and K is the dielectric constant of the dielectric layer. The energy storage capacity U of the capacitor may be approximated as:

U=½CV_{bd}² (4)

which may be rewritten using equations (2) and (3) as:

U=½κε₀AE_{bd}² (5)

The energy storage capacity U is determined by the dielectric constant κ, the area A, and the breakdown field E_{bd}. By appropriate engineering, a capacitor or capacitor bank may be designed to have any desired energy storage capacity U. By way of example, and not by way of limitation, given the above ranges for the dielectric constant κ, electrode area A, and breakdown field E_{bd} a capacitor in accordance with aspects of the present disclosure may have an energy storage capacity U ranging from about 500 Joules to about 2×10¹⁶ Joules.

For a dielectric constant κ ranging, e.g., from about 100 to about 1,000,000 and constant breakdown field E_{bd} between, e.g., about 0.1 and 0.5 V/nm, a capacitor of the type described herein may have a specific energy capacity per unit mass ranging from about 10 W·h/kg up to about 100,000 W·h/kg, though implementations are not so limited.

Aspects of the present disclosure include meta-capacitors that are coiled, e.g., as depicted in FIG. 1B. In this example, a meta-capacitor 20 comprises a first electrode 21, a second electrode 22, and a meta-dielectric material layer 23 of the type described hereinabove disposed between said first and second electrodes. The electrodes 21, 22 may be made of a metal, such as copper, zinc, or aluminum or other conductive material and are generally planar in shape. In one implementation, the electrodes and meta-dielectric material layer 23 are in the form of long strips of material that are sandwiched together and wound into a coil along with an insulating material, e.g., a plastic film such as polypropylene or polyester to prevent electrical shorting between the electrodes 21, 22. Examples of such coiled capacitor energy storage devices are described in detail in commonly-assigned U.S. patent application Ser. No. 14/752,600, filed Jun. 26, 2015, the entire contents of which are incorporated herein by reference.

In order that the invention may be more readily understood, reference is made to the following examples, which are intended to be illustrative of the invention, but are not intended to be limiting the scope.

### Example 1

This Example describes synthesis of one type of Sharp polymer according following structural scheme:

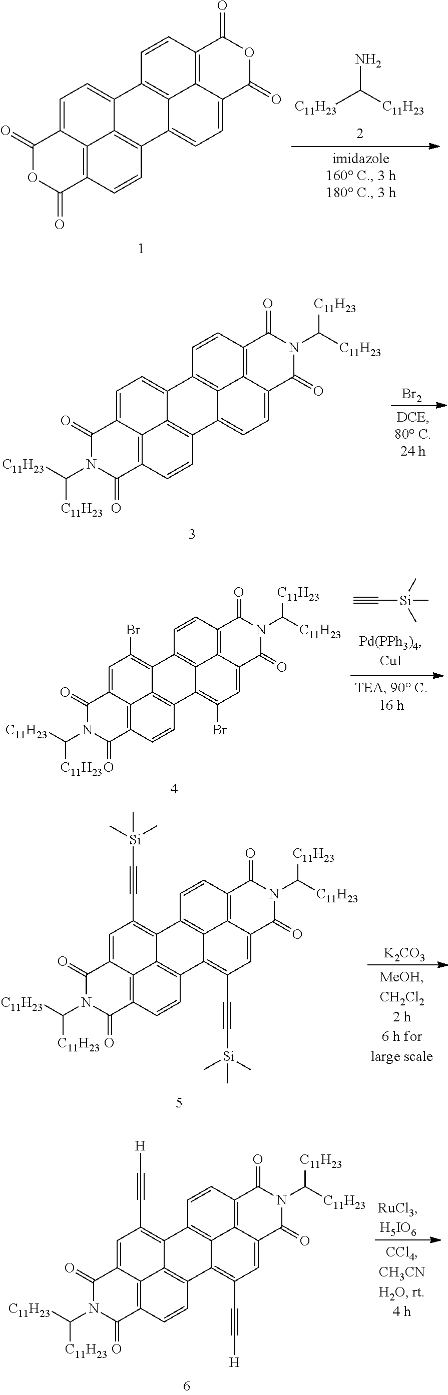

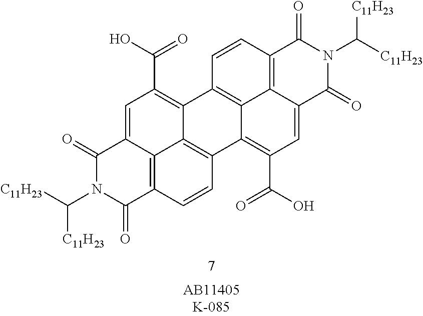

The process involved in the synthesis in this example may be understood in terms of the following five steps.

### a) First Step:

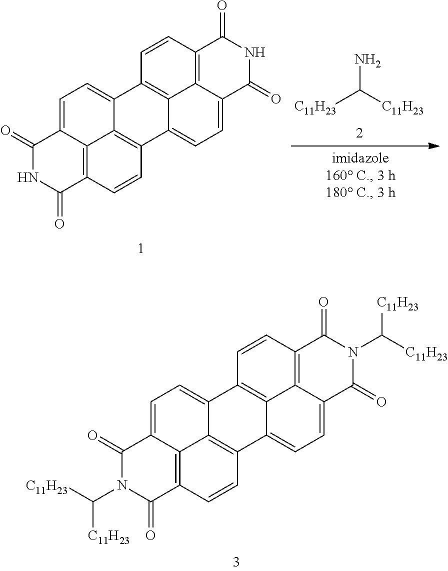

Anhydride 1 (60.0 g, 0.15 mol, 1.0 eq), amine 2 (114.4 g, 0.34 mol, 2.2 eq) and imidazole (686.0 g, 10.2 mol, 30 eq to 2) were mixed well into a 500 mL of round-bottom flask equipped with a bump-guarder. The mixture was degassed three times, stirred at 160° C. for 3 hr, 180° C. for 3 hr, and cooled to rt. The reaction mixture was crushed into water (1000 mL) with stirring. Precipitate was collected with filtration, washed with water (2×500 mL), methanol (2×300 mL) and dried on high vacuum. The crude product was purified by flash chromatography column (CH₂Cl₂/hexane=1/1) to give 77.2 g (48.7%) of the desired product 3 as an orange solid. ¹H NMR (300 MHz, CDCl₃) δ 8.65-8.59 (m, 8H), 5.20-5.16 (m, 2H), 2.29-2.22 (m, 4H), 1.88-1.82 (m, 4H), 1.40-1.13 (m, 64H), 0.88-0.81 (t, 12H). Rf=0.68 (CH₂Cl₂/hexane=1/1).

### b) Second Step:

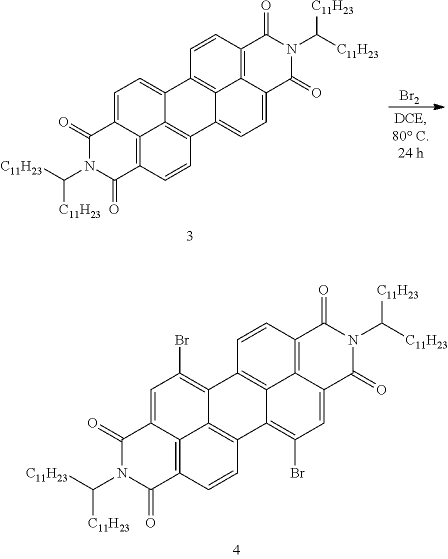

To a solution of the diimide 3 (30.0 g, 29.0 mmol, 1.0 eq) in dichloroethane (1500 mL) was added bromine (312.0 g, 1.95 mol, 67.3 eq). The resulting mixture was stirred at 80° C. for 36 hr, cooled, washed with 10% NaOH (aq, 2×1000 mL), water (100 ml), dried over Na₂SO₄, filtered and concentrated. The crude product was purified by flash chromatography column (CH₂Cl₂/hexanes=1/1) to give 34.0 g (98.2%) of the desired product 4 as a red solid. ¹H NMR (300 MHz, CDCl₃) δ 9.52 (d, 2H), 8.91 (bs, 2H), 8.68 (bs, 2H), 5.21-5.13 (m, 2H), 2.31-2.18 (m, 4H), 1.90-1.80 (m, 4H), 1.40-1.14 (m, 64H), 0.88-0.81 (t, 12H). Rf=0.52 (CH₂Cl₂/hexanes=1/1).

### c) Third Step

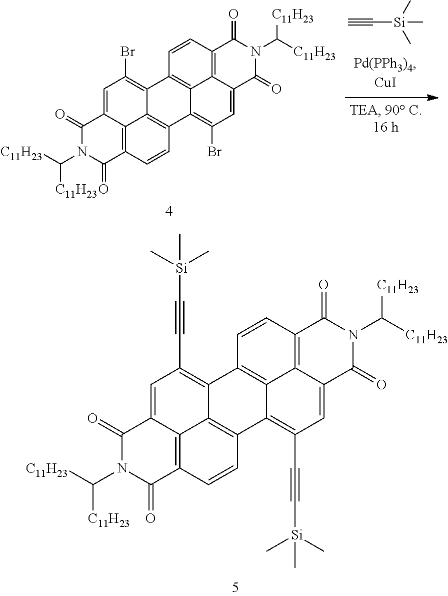

To a solution of the di-bromide 4 (2.0 g, 1.68 mmol, 1.0 eq) in triethylamine (84.0 mL) was added CuI (9.0 mg, 0.048 mmol, 2.8 mol %) and (trimethylsilyl)acetylene (80.49 g, 5.0 mmol, 3.0 eq). The mixture was degassed three times. Catalyst Pd(PPh₃)₄ (98.0 mg, 0.085 mmol, 5.0 mol %) was added. The mixture was degassed three times, stirred at 90° C. for 24 hr, cooled, passed through a pad of Celite, and concentrated. The crude product was purified by flash chromatography column (CH₂Cl₂/hexane=1/1) to give 1.8 g (87.2%) of the desired product 5 as a dark-red solid. ¹H NMR (300 MHz, CDCl₃) δ 10.24-10.19 (m, 2H), 8.81 (bs, 2H), 8.65 (bs, 2H), 5.20-5.16 (m, 2H), 2.31-2.23 (m, 4H), 1.90-1.78 (m, 4H), 1.40-1.15 (m, 72H), 0.84-0.81 (t, 12H), 0.40 (s, 18H). Rf=0.72 (CH₂Cl₂/hexane=1/1).

### d) Fourth Step

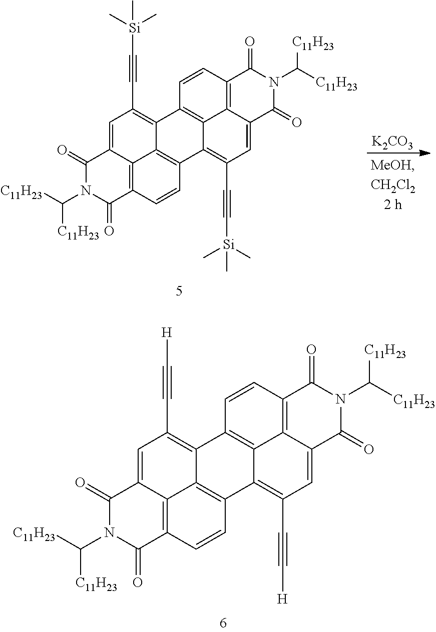

To a solution of diimide 5 (1.8 g, 1.5 mmol, 1.0 eq) in a mixture of MeOH/DCM (40.0 mL/40.0 mL) was added K₂CO₃ (0.81 g, 6.0 mmol, 4.0 eq). The mixture was stirred at room temperature for 1.5 hr, diluted with DCM (40.0 mL), washed with water, brine, dried over Na₂SO₄, filtered and concentrated. The crude product was purified by flash chromatography column (CH₂Cl₂) to give 1.4 g (86.1%) of the desired product 6 as a dark-red solid. H NMR (300 MHz, CDCl₃) δ 10.04-10.00 (m, 2H), 8.88-8.78 (m, 2H), 8.72-8.60 (m, 2H), 5.19-5.14 (m, 2H), 3.82-3.80 (m, 2H), 2.31-2.23 (m, 4H), 1.90-1.78 (m, 4H), 1.40-1.05 (m, 72H), 0.85-0.41 (t, 12H). Rf=0.62 (CH₂Cl₂).

### e) Fifth Step

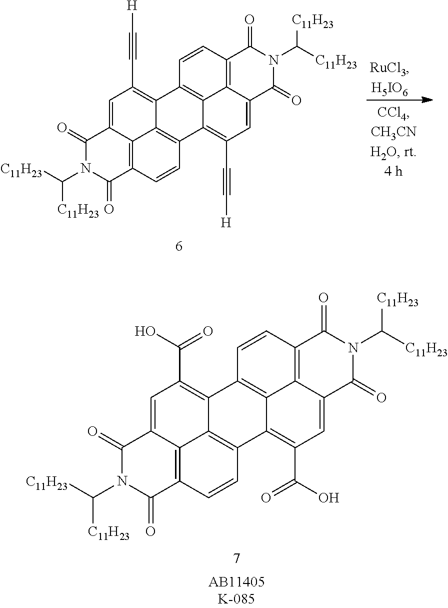

To a suspension of alkyne 6 (1.4 g, 1.3 mmol, 1.0 eq) in a mixture of CCl₄/CH₃CN/H₂O (6 mL/6 mL/12 mL) was added periodic acid (2.94 g, 12.9 mmol, 10.0 eq) and RuCl₃ (28.0 mg, 0.13 mmol, 10 mol %). The mixture was stirred at room temperature under nitrogen for 4 hours, diluted with DCM (50 mL), washed with water, brine, dried over Na₂SO₄, filtered and concentrated. The crude product was purified by flash chromatography column (10% MeOH/CH₂Cl₂) to give 1.0 g (68.5%) of the desired product 7 as a dark-red solid. ¹H NMR (300 MHz, CDCl₃) δ 8.90-8.40 (m, 6H), 5.17-5.00 (m, 2H), 2.22-2.10 (m, 4H), 1.84-1.60 (m, 4H), 1.41-0.90 (m, 72H), 0.86-0.65 (t, 12H). Rf=0.51 (10% MeOH/CH₂Cl₂).

### Example 2

This Example describes synthesis of a Sharp polymer according following structural scheme:

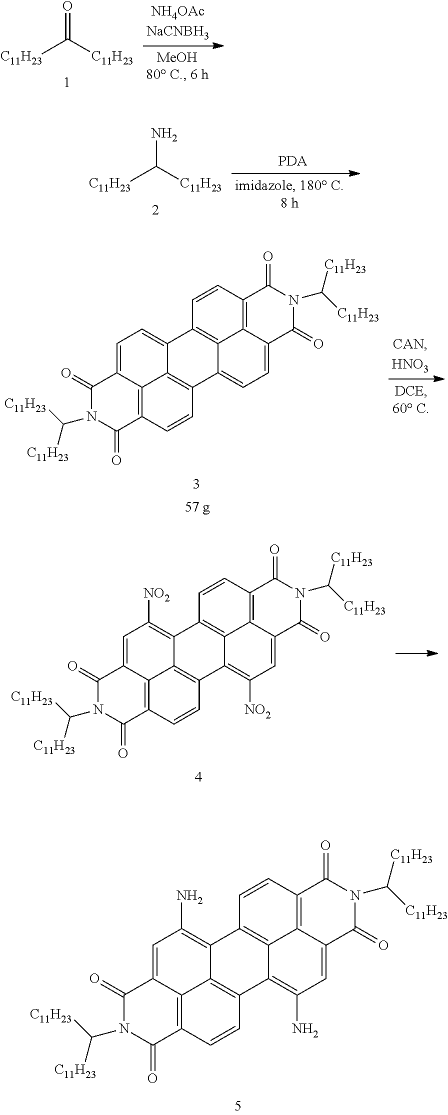

The process involved in the synthesis in this example may be understood in terms of the following four steps.

### a) First Step:

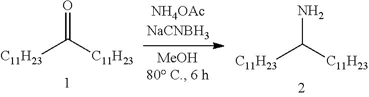

To a solution of the ketone 1 (37.0 g, 0.11 mol, 1.0 eq) in methanol (400 mL) was added ammonium acetate (85.3 g, 1.11 mol, 10.0 eq) and NaCNBH₃ (28.5 g, 0.44 mol, 4.0 eq) in portions. The mixture was stirred at reflux for 6 hours, cooled to room temperature and concentrated. Sat. NaHCO₃ (500 mL) was added to the residue and the mixture was stirred at room temperature for 1 hour. Precipitate was collected by filtration, washed with water (4×100 mL), dried on a high vacuum to give 33.6 g (87%) of the amine 2 as a white solid.

### b) Second Step:

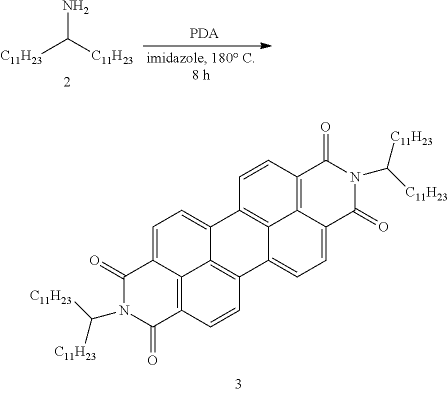

Mixed well the amine 2 (20.0 g, 58.7 mmol, 2.2 equ), 3,4,9,10-perylenetetracarboxylic dianhydride (10.5 g, 26.7 mmol, 1.0 eq) and imidazole (54.6 g, 0.80 mmol, 30 eq to diamine) into a 250 mL round-bottom flask equipped with a rotavap bump guard. The mixture was degased (vacuum and fill with N₂) three times and stirred at 160° C. for 6 hrs. After cooling to rt, the reaction mixture was crushed into water (700 mL), stirred for 1 hr, and filtered through a filter paper to collected precipitate which was washed with water (3×300 mL) and mthanol (3×300 mL), dried on a high vacuum to give 23.1 g (83.5%) of the diamidine 3 as a orange solid. Pure diamidine 3 (20.6 g) was obtained by flash chromatography column (DCM/hexanes=1/1).

### c) Third Step:

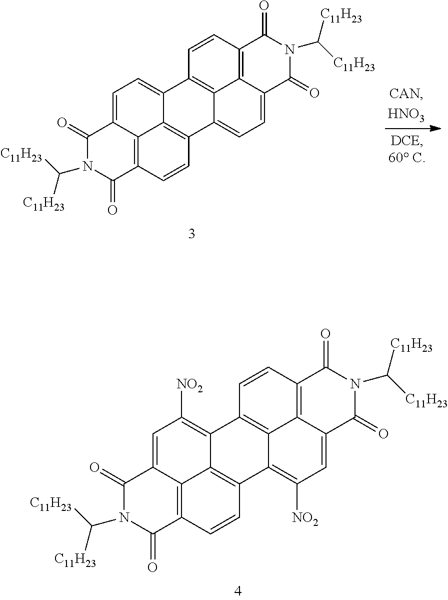

To DCE (2.0 L) was added compound 3 (52.0 g, 50.2 mmol, 1.0 eq), acetic acid (500 mL) and fuming nitric acid (351.0 g, 5.0 mol, 100.0 eq) with caution. To the mixture was added ammonium cerium(IV) nitrate (137.0 g, 0.25 mol, 5.0 eq). The reaction was stirred at 60° C. for 48 hrs. After cooling to rt, the reaction mixture was crushed into water (1.0 L). The organic phase was washed with water (2×1.0 L), saturated NaHCO₃ solution (1×1.0 L) and brine (1×1.0 L), dried over sodium sulfate, filtered and concentrated. The residue was purified with column chromatography to give 46.7 g (82%) of compound 4 as a dark red solid. ¹H NMR (300 MHz, CDCl₃) δ 0.84 (t, 12H), 1.26 (m, 72H), 1.83 (m, 4H), 2.21 (m, 4H), 5.19 (m, 2H), 8.30 (m, 2H), 8.60-8.89 (m, 4H).

### d) Fourth Step:

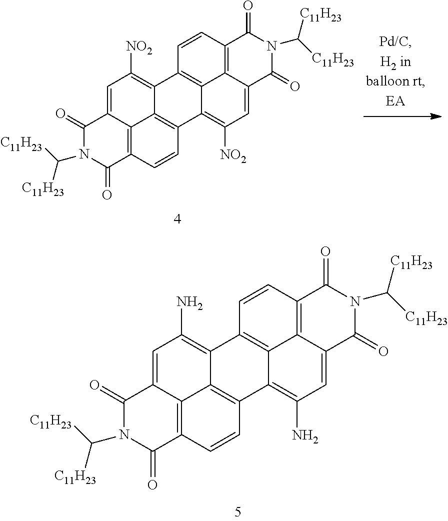

A mixture of compound 4 (25 g, 22.2 mmol, 1.0 eq) and Pd/C (2.5 g, 0.1 eq) in EtOAc (125.0 mL) was stirred at room temperature for 1 hour. The solid was filtered off (Celite) and washed with EtOAc (5 mL×2). The filtrate was concentrated to afford the compound 5 (23.3 g, 99%) as a dark blue solid. ¹H NMR (300 MHz, CDCl₃) δ 0.84 (t, 12H), 1.24 (m, 72H), 1.85 (m, 4H), 2.30 (m, 4H), 5.00 (s, 2H), 5.10 (s, 2H), 5.20 (m, 2H), 7.91-8.19 (dd, 2H), 8.40-8.69 (dd, 2H), 8.77-8.91 (dd, 2H).

While the above is a complete description of the preferred embodiment of the present invention, it is possible to use various alternatives, modifications and equivalents. Therefore, the scope of the present invention should be determined not with reference to the above description but should, instead, be determined with reference to the appended claims, along with their full scope of equivalents. Any feature described herein, whether preferred or not, may be combined with any other feature described herein, whether preferred or not. In the claims that follow, the indefinite article “A”, or “An” refers to a quantity of one or more of the item following the article, except where expressly stated otherwise. As used herein, in a listing of elements in the alternative, the word “or” is used in the logical inclusive sense, e.g., “X or Y” covers X alone, Y alone, or both X and Y together, except where expressly stated otherwise. Two or more elements listed as alternatives may be combined together. The appended claims are not to be interpreted as including means-plus-function limitations, unless such a limitation is explicitly recited in a given claim using the phrase “means for.”

1-16. (canceled)

17. A meta-capacitor, comprising two metal electrodes; and a meta-dielectric film between the two electrodes, the meta-dielectric film comprising composite molecules with a resistive envelope built with oligomers having a composition of hydrocarbon (saturated and/or unsaturated), fluorocarbon, siloxane, and/or polyethyleneglycol as linear or branched chains and a polarizable core molecular fragment inside the resistive envelope, wherein the polarizable core has an electronic or ionic type of polarizability provided by electronic conductivity of the core molecular fragment or limited mobility of ionic parts of the core molecular fragment.

18. A meta-capacitor, comprising:
first and second electrodes and a meta-dielectric material disposed between the first and second electrodes, wherein the meta-dielectric material is a Sharp polymer characterized by polarizability and resistivity that is having a following general structural formula: 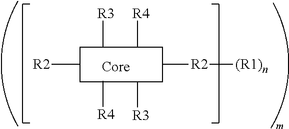
wherein Core is an aromatic polycyclic conjugated molecule comprising a flat anisometric form and self-assembling by pi-pi stacking in a column-like supramolecule,
wherein R1 is substitute providing solubility of the organic compound in a solvent,
wherein n is number of substitutes R1 which is equal to 0, 1, 2, 3, 4, 5, 6, 7 or 8,
wherein R2 is electrically resistive substitute located in terminal positions, which provides resistivity to electric current and comprises hydrocarbon (saturated and/or unsaturated), fluorocarbon, siloxane, and/or polyethyleneglycol as linear or branched chains,
wherein R3 and R4 are substitutes located on lateral positions (terminal and/or bay positions) comprising one or more ionic groups from a class of ionic compounds that are used in ionic liquids connected to the aromatic polycyclic conjugated molecule (Core) directly or via a connecting group, and
wherein m is number of the aromatic polycyclic conjugated molecules in the column-like supramolecule which is in the range from 3 to 100,000.

19. The meta-capacitor of claim 18, wherein the aromatic polycyclic conjugated molecule (Core) comprises rylene fragments.

20. The meta-capacitor of claim 18, wherein the aromatic polycyclic conjugated molecule comprises an electro-conductive oligomer selected from the group of a phenylene, thiophene, or a polyacene quinine radical oligomer or a combination or two or more of these.

21. The composite organic compound of claim 20, wherein the electro-conductive oligomer is selected from structures 22 to 30 wherein I=2, 3, 4, 5, 6, 7, 8, 9, 10, 11 or 12, Z is ═O, ═S or ═NR5, and R5 is selected from the group consisting of unsubstituted or substituted C₁-C₁₈alkyl, unsubstituted or substituted C₂-C₁₈alkenyl, unsubstituted or substituted C₂-C₁₈alkynyl, and unsubstituted or substituted C₄-C₁₈aryl:

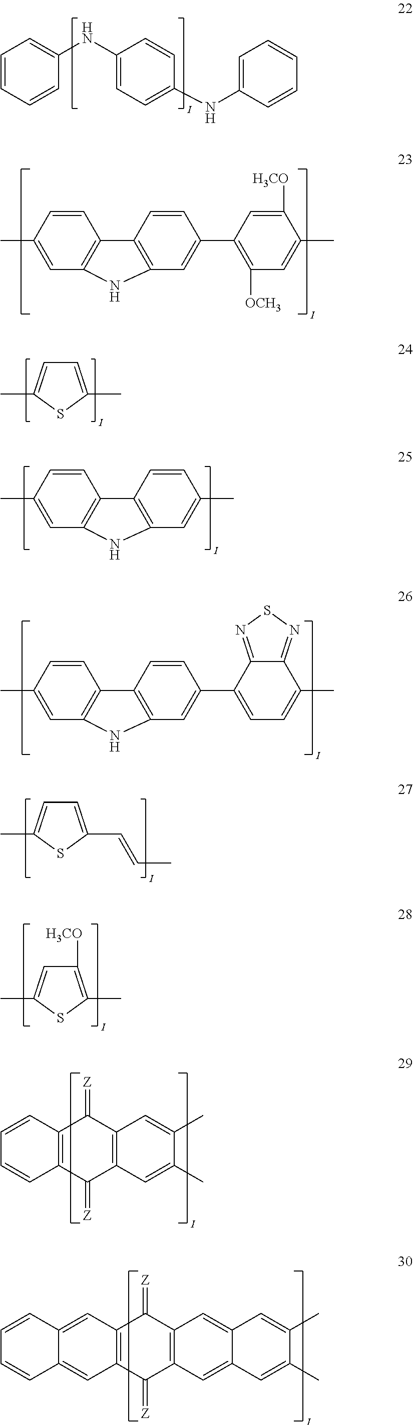

22. The meta-capacitor of claim 18, wherein the substitute providing solubility (R1) of the composite organic compound is selected from the group of alkyl, aryl, substituted alkyl, substituted aryl, fluorinated alkyl, chlorinated alkyl, branched and complex alkyl, branched and complex fluorinated alkyl, branched and complex chlorinated alkyl groups, and any combination thereof, and wherein the alkyl group is selected from methyl, ethyl, propyl, n-butyl, iso-butyl and tert-butyl groups, and the aryl group is selected from phenyl, benzyl and naphthyl groups or siloxane, and/or polyethyleneglycol as linear or branched chains.

23. The meta-capacitor of claim 19, wherein the substitute providing solubility (R1) of the composite organic compound is C_{X}Q_{2X+1}, where X≥1 and Q is hydrogen (H), fluorine (F), or chlorine (Cl).

24. The meta-capacitor of claim 18, wherein the solvent is selected from benzene, toluene, xylenes, acetone, acetic acid, methylethylketone, hydrocarbons, chloroform, carbontetrachloride, methylenechloride, dichloroethane, chlorobenzene, alcohols, nitromethane, acetonitrile, dimethylformamide, 1,4-dioxane, tetrahydrofuran (THF), methylcyclohexane (MCH), and any combination thereof.

25. The meta-capacitor of claim 18, wherein at least one electrically resistive substitute (R2) is selected from the group of alkyl, aryl, substituted alkyl, substituted aryl, fluorinated alkyl, chlorinated alkyl, branched and complex alkyl, branched and complex fluorinated alkyl, branched and complex chlorinated alkyl groups, and any combination thereof, and wherein the alkyl group is selected from methyl, ethyl, propyl, n-butyl, iso-butyl and tert-butyl groups, and the aryl group is selected from phenyl, benzyl and naphthyl groups or siloxane, and/or polyethyleneglycol as linear or branched chains.

26. The meta-capacitor of claim 18, wherein at least one electrically resistive substitute (R2) is C_{X}Q_{2X+1}, where X≥1 and Q is hydrogen (H), fluorine (F), or chlorine (Cl).

27. The meta-capacitor of claim 18, the substitute R3 and/or R4 is connected to the aromatic polycyclic conjugated molecule (Core) via at least one connecting group.

28. The meta-capacitor of claim 27, wherein the at least one connecting group is selected from the list comprising the following structures: 31-41, where W is hydrogen (H) or an alkyl group:

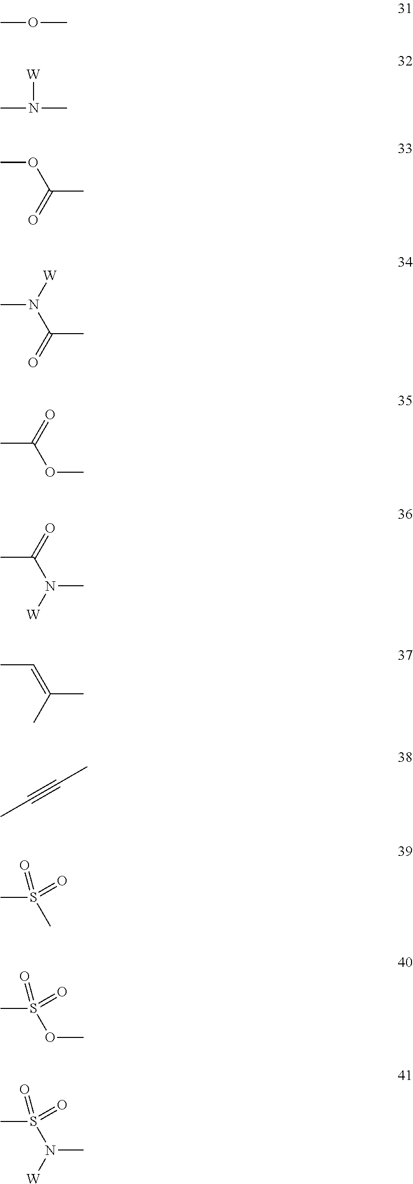

29. The meta-capacitor of claim 18, wherein the substitute R3 and/or R4 is connected to the aromatic polycyclic conjugated molecule (Core) via at least one connecting group.

30. The meta-capacitor of claim 29, wherein the at least one connecting group is selected from the group of CH₂, CF₂, SiR₂O, CH₂CH₂O, wherein R is selected from the list comprising H, alkyl, and fluorine.

31. The meta-capacitor of claim 18, wherein the one or more ionic groups include at least one ionic group selected from the list comprising [—NR₃]⁺, [—PR₃]⁺ as cation and [—CO₂]⁻, [—SO₃]⁻, [—SR₅]⁻, [—PO₃R]⁻, [—PR₅]⁻ as anion, wherein R is selected from the list comprising H, alkyl, and fluorine.
